(19) United States
(12) Patent Application Publication
(10) Pub. No.: US 2009/0048590 A1
(43) Pub. Date: Feb. 19, 2009

(54) SYSTEMS AND METHODS FOR TREATING NAIL-BED FUNGUS THROUGH APPLICATION OF HEAT

(75) Inventors: Robert A. Conrad, Spring, TX (US); Charles Conrad, Spring, TX (US); Walter V. Klemp, Houston, TX (US)

Correspondence Address: FULBRIGHT & JAWORSKI L.L.P, 2200 ROSS AVENUE, SUITE 2800, DALLAS, TX, 75201-2784, US

(73) Assignee: Tyrell, Inc., Houston, TX (US)

(21) Appl. No.: 11/839,167

(22) Filed: Aug. 15, 2007

(51) Int. Cl. A61F 7/00; A61B 18/04

(52) U.S. Cl. 606/28; 607/112

(57) ABSTRACT

A regulated heat source is described that can be applied to the nail bed in order to accelerate the death of the organisms know to contribute to nail bed infections

### TECHNICAL FIELD

The present invention relates to methods and systems for treatment of nail-bed infections. More specifically, the present invention relates to methods and systems for treatment of nail-bed fungus involving the application of thermal energy.

### BACKGROUND OF THE INVENTION

Nail disorders comprise about ten percent of all skin disorders and fungal nail infections (onychomycosis) account for approximately fifty percent of all nail problems. It is estimated that two to three percent of the United States population has onychomycosis. The disorder is twice as frequent among men and increases with age.

Onychomycosis, which causes the nails to become thickened, hard to cut and often painful, is worsened by moisture, warmth, trauma, communal bathing, and other activities that lead to the exposure to fungi. Common complaints of the disorder are pain, deformed nails, and interference with daily activities such as walking, typing, or playing a musical instrument. Those who suffer from onychomycosis cite a substantial negative effect on their quality of life. Fingernail infections have a significant effect on the life of infected individuals. Embarrassment may prevent patients from taking part in social situations because they feel unwilling to show their hands or feet.

While Onychomycosis typically refers to the invasion of the nail bed by a fungus, the infection may be due to a fungus, a mold, or non-dermatophyte yeast. The group of fungi most commonly responsible for causing infection of the nail bed are known as dermatophytes and include the genera Trichophyton, Microsporum, and Epidermophyton. Infections can also be caused by Candida species, which are yeasts. The most common Candida species causing infection is Candida alicats. The most common non-dermatophyte molds associated with nail disease are Scopulariopsis, Scytalidium, Fulsarium, Aspergillus and Onychocola canadensis.

Until now, the methods and devices for treating onychomycosis have included treatment with topical antifungal agents, oral antifungal drugs, thinning or partial removal of the nail, and permanent removal of the nail. Often, these techniques take up to year to no longer see the symptoms of onychomycosis.

It has been demonstrated that the application of heat at various time and temperature combinations reliably kills the P. acnes and Staphylococcus aureus bacteria, as well as the HSV1 virus. The necessary temperature range to kill bacteria is generally above 47 degrees Celsius. but below the bum or discomfort threshold for human skin. Depending on the area of skin and the area of surface contact, this upper threshold is in the range of 51 degrees Celsius. A treatment method using a rapid, transient heat application, has been suggested for the treatment of onychomycosis (Chato, J. C., Thermal Therapy of Toe Nail Fungus, Int'l Mech. Eng. Congress and Exposition 2000, Nov. 11-16, 2000, Orlando, Fla.). This method employed the use of hot water, heated to a temperature of 50 degrees Celsius, applied near the base of the nail for a period of 4 to 5 seconds repeated three times, approximately 15 seconds apart. Because of the thick nail plate and possible separation of the nail plate from the nail bed, this rapid, transient heat application is unlikely to have any appreciable effect on the fungal infection.

### BRIEF SUMMARY OF THE INVENTION

This invention relates to the use of a regulated heat source that can be applied to a nail bed infection, such as onychomycosis, in order to initiate and/or accelerate the death of the bacteria, dermatophyte, mold, virus, or non-dermatophyte yeast causing the nail bed infection and thereby speed the recovery process.

In one embodiment, a device for treating nail bed fungus is described. The device includes a thermal delivery surface designed to be placed in contact with the nail plate. The thermal delivery surface is adapted to transfer thermal energy from the device to the nail bed. There may also be a thermal transfer medium that is attached to the nail plate. The thermal transfer medium is designed to be flexible, deformable, and highly conductive and is used to improved the transfer of thermal energy from the thermal delivery surface to the nail bed. Thermal transfer from the thermal deliver surface may further be enhanced by a spring tensioning system used to apply a downward pressure on the thermal delivery surface according to embodiments of the invention. In situations were the nail plate has separate from the nail bed, a thermal gel can be applied to the air gap formed between the nail plate and nail bed. The combination of the thermal delivery surface, thermal transfer medium, thermal gel, and downward force of the spring tensioning system provide greatly improved thermal energy transfer.

In another embodiments the thermal delivery surface is attached to the nail plate using a flexible strap. The flexible strap may be made of an elastic material and a fastening system is used to apply the thermal transfer surface to the nail plate. The flexible nature of the strap allows tension to be applied to the strap to produce a downward pressure on the thermal transfer surface. As discussed above, the transfer of thermal energy can also be improved by the use of a thermal transfer medium and thermal gel.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a perspective view of an embodiment of a treatment device according to the present invention;

FIG. 2 shows a perspective view of an embodiment of a thermal transfer kit for use with a treatment device according to the present invention;

FIGS. 3A and 3B shows a nail bed, a nail plate, and an air gap between the two faces;

FIGS. 4A and 4B shows the filling of the air gap of FIGS. 3A and 3B and clamping of the thermal gel pack according to an embodiment of the present invention;

FIG. 5 shows a perspective view of an embodiment of a flexible thermal step for use with a treatment device according to the present invention;

FIG. 6 shows a simplified block diagram of the major electrical components of an embodiment of the treatment device of FIG. 1;

FIG. 7 is a diagram illustrating the control functionality of the firmware used in an embodiment of the present invention; and

FIG. 8 shows a state diagram illustrating the operation of a treatment device according to an embodiment of the present invention.

### DETAILED DESCRIPTION OF THE INVENTION

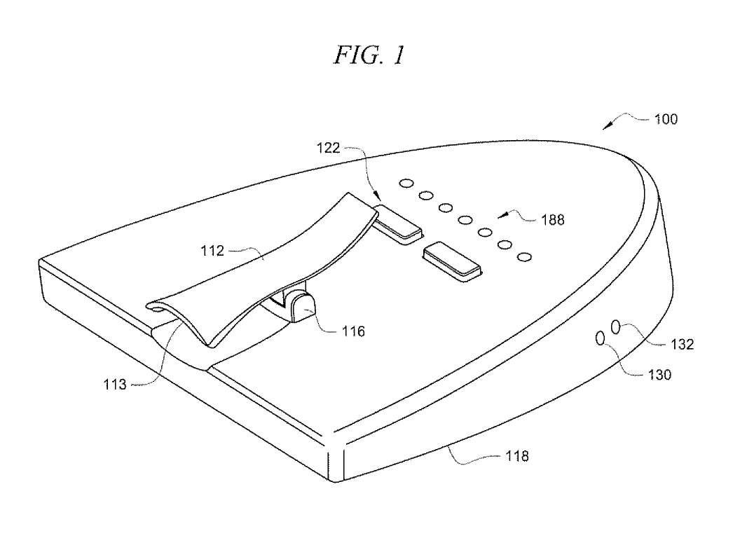

An embodiment of a device for treating or preventing nail bed infections is shown in FIG. 1. Treatment device 100 of a preferred embodiment operates to transfer thermal energy to the nail plate at a set temperature for a set period of time. The set temperature and set period of time can be varied to accommodate different disease conditions and patient tolerance levels. Alternatively, treatment device 1 00 can be can configured with permanent time and temperature settings selected to treat a specific type of nail bed infection. However, treatment device 100 preferably should be capable of heating a treatment surface to a temperature, as close as can be controlled using electrical heating with temperature feedback, between about 46 degrees Celsius and about 68 degrees Celsius and sustaining one or more temperatures for between about 10 seconds and about 30 minutes. Although thermal damage generally occurs when human skin is heated to a temperature of approximately 66 degrees Celsius or higher, an interface heated to this temperature or a higher temperature can nevertheless deliver an therapeutically effective amount of heat to the nail plate without resulting in thermal damage, depending on the amount of thermal energy delivered over a particular surface area and how readily the thermal energy is dissipated by the heated tissue.

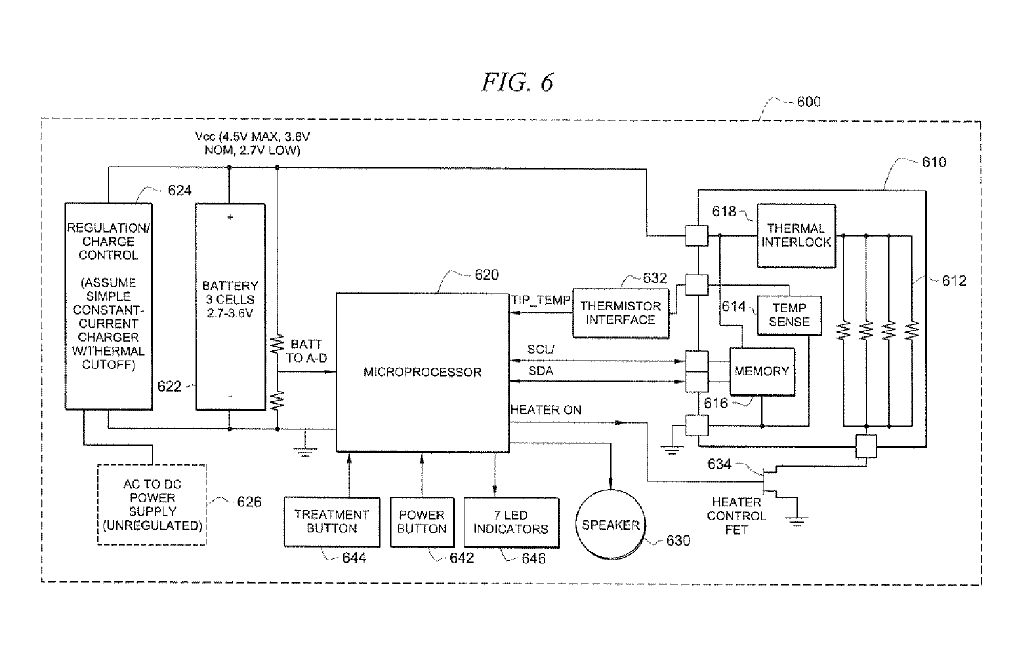

Treatment device 100 comprises a nail cover 112 connected by spring tensioning system 116 to housing 118. Thermal delivery surface 113 located under nail cover 112 also preferably includes a temperature sensor, not shown. Housing 118 comprises a protective cover to hold the internal electrical components of treatment device 100 (a preferred embodiment of which is shown and described below with respect to FIG. 6) and a user interface 122. By means of a user interface 122, the user may activate and monitor the device.

Nail cover 112, thermal delivery surface 113, and spring tensions system 116 are replaceable according to embodiments of the invention. Nail cover 112 and thermal delivery surface 113 can be changed as necessary to accommodate the various differences in the size of the patent's digits (length of nail, width of nail, thickness of digit). Additionally, spring tensioning system 116 can be exchanged with various spring strengths as necessary for patient comfort.

Thermal delivery surface 113 contains a heating element which is electrically connected to treatment device 100. Examples of heating elements include resistance heaters, etch foil heaters, silicone rubber heaters, heat cables, fiberglass heating tapes, wire wound flexible heaters, electric heating tape, cable heaters, tape heaters. Treatment device 100 provides electrical current to the heating element of thermal delivery surface 113 by means of rechargeable batteries. The heating element of thermal delivery surface 113 produces heat through electrical resistance, which, in turn, is monitored by treatment device 100. The temperature of thermal delivery surface 113 is monitored by a temperature sensor, which may be a thermistor or other electrical device that measures or monitors heat. Treatment device 100 is able to adjust the power provided to thermal delivery surface 113 so as to maintain thermal delivery surface 113 at or near a set temperature chosen for the treatment.

As noted above, housing 118 holds the internal electrical components and the power source, such as rechargeable batteries. While treatment device 100 is described as using rechargeable batteries as the preferred power source, any suitable power source may be used, including receiving power from an ordinary household power plug and socket connection. A speaker, not shown, is also housed in housing 118.

Treatment device 100 of the illustrated embodiment includes a battery charge port 130 and a data port 132. Battery charge port 130 is used to plug in a charger to charge the internal batteries or, in some embodiments, to power the device from line power. Data port 132 allows treatment device 100 to communicate with another device, such as a computer or PDA, and allows the internal electrical components to receive new programs or new data to be used in treatment device 100. Data that can be communicated from the device to a computer or PDA includes, but is not limited to, number of treatments, duration of treatment, temperature of treatment, date and time of treatment. Additionally, a computer or PDA can be used to chance treatment settings such as duration, temperature, etc. Although the embodiment shown in FIG. 1 contains battery charge port 130 and data port 132 on the side of housing 118, battery charge port 130 and data port 132 may be found in another location of housing 118.

User interface 120 of the illustrated embodiment includes power button 122, treatment button 124, and light emitting diodes (LEDs) 188. Power button 122 of one embodiment is used to turn treatment device 110 on and off. Treatment button 124 of the aforementioned embodiment is used to initiate and/or cancel treatments. Treatment button 124 can include LEDs 188 that indicate whether treatment device 10 is ready to begin a treatment. While the illustrated embodiment is shown using LEDs as a display, any display technology such as LCDs or other display may be used without departing from the concepts described herein. For example. LEDs 188 could include an amber light to indicate that the device is not ready to begin a treatment and a green light to indicate that treatment device 100 is ready to begin a treatment. Treatment device 100 may comprise additional LEDs not shown to provide additional visual information to the user, such as the charge remaining in the battery and any other information which may be useful or interesting to the user. The speaker can be used to provide audible information to the user such as the amount of time remaining in the treatment, an error condition, low battery charge, and any other audible information that might be useful or interesting to the user. Additionally, other user interlaces may also be used, including touch screens, slide controls, keyboards, light pens, microphones, speech recognition, pointing devices (mouse, track ball, etc.), and gesture devices.

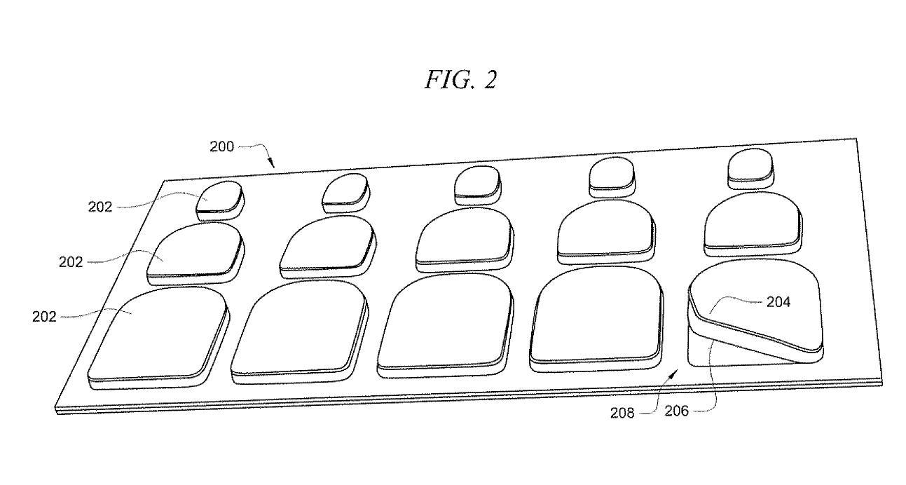

Referring now to FIG. 2, an embodiment of a thermal transfer kit 200 is shown. The thermal transfer kit includes thermal transfer medium to aid in the transfer of heat from the thermal delivery surface to the nail plate. The thermal transfer kit of the illustrated embodiment includes thermal gel packs 202, which, when applied to the nail plate, transfers thermal energy from thermal delivery surface 113 of FIG. 1 sufficient to combat the bacteria, viruses, fungi, mold, and non-dermatophyte yeast known to contribute to nail bed infections. Thermal gel pack 202 of a preferred embodiment is flexible and conforms to the shape of the nail plate and has an adhesive surface 206, which, when removed from carrier 208 and applied to the nail plate holds the thermal gel pack on the nail plate. Additionally or alternatively, thermal gel packs 202 may include adhesive surface 204 to hold the gel pack to thermal delivery surface 113 of FIG. 1. Thermal gel pack 202 may be a customizable gel pack which can be made to generally or specifically fit the nail bed of a particular patient.

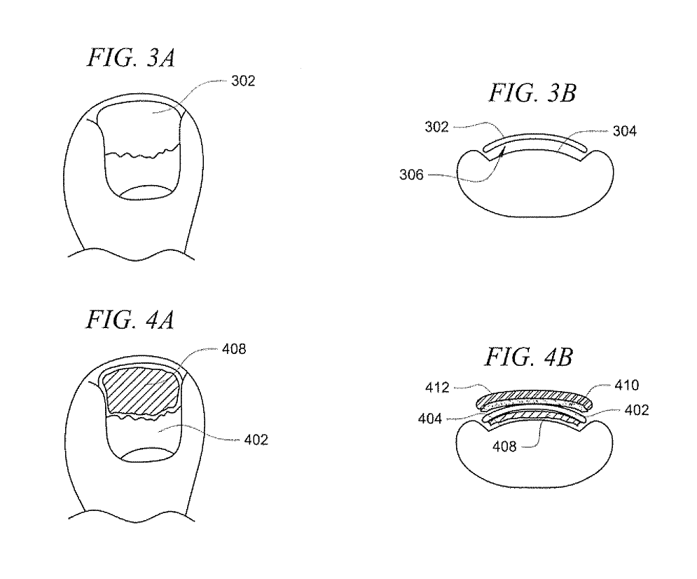

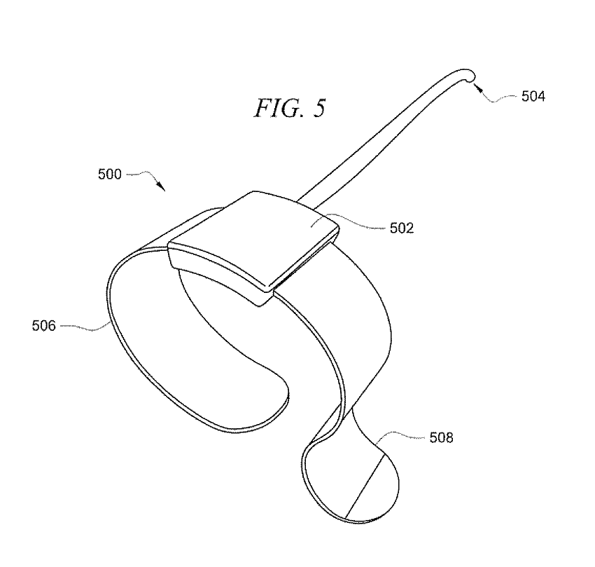

As shown in FIGS. 3A and 3B, the nail bed infection can often cause nail plate 302 to separate from nail bed 304, leaving an air gap 306 between the two surfaces. This air gap between nail plate 302 and nail bed 304 lowers the heat transferred to nail bed 304. Referring now to FIG. 4, air gap 304 of FIG. 3B may be filled with thermal gel 408 to improve heat transfer. The nail plate 402 may be gently lifted to allow thermal gel 408 to be introduced and fill the air gap between nail plate 402 and nail bed 404. Thermal gel 408 may be introduced using a syringe and needle, a tube with an applicator tip, sprayed in, or using any other means to fill air gap 304 of FIG. 3A. To further improve the heat transfer to the nail bed, thermal gel 408 is used in combination with thermal gel pack 410 and thermal delivery surface 412.

In operation according to a preferred embodiment, thermal gel pack 406 is applied to the nail plate 402, air gap 304 of FIG. 3A is filled with thermal gel 408, and the nail to be treated is placed in treatment device 100 of FIG. 1. Nail cover 112 of FIG. 1 applies a slight downward pressure on nail plate 402. The combination of filling the air gap with thermal gel 408, applying thermal gel pack 412 to nail plate 402, and applying a slight downward pressure using the clamping mechanism of nail cover 414 will noticeably improve heat transfer to the nail bed. Further, an anti-fungal ingredient such as clotrimazole, ciclopirox, econazole nitrate, ketoconazole, myconazole nitrate, ketoconazole, or trebinafine hydrochloride may be added to the thermal gel formulation to improve the kill rate.

Now referring to FIG. 5, an alternate means of applying a dose of thermal energy to the nail bed is by use of flexible strap 500 comprising a soft, flexible material designed to control tension and hold thermal delivery surface 502 against the nail plate or a thermal gel pack. Additionally, flexible strap 500 may be used in combination with thermal transfer media 406 and thermal gel 408 of FIG. 4 to improve the heat transfer to the nail bed. Flexible strap 500 comprises a soft, flexible material 506 designed to conform to the irregular shapes of nail plates and a fastening means 508 such as an adhesive fastening system or a mechanical fastening system. The use of adhesive fastening systems are well known in the art. Examples of these adhesive tape fastening systems are described in U.S. Pat. No. 3,848,592 entitled “Tape Fastening Systems for Disposable Diaper” issued to Kenneth B. Buell on Nov. 19, 1974; and U.S. Pat. No. 5,413,568 entitled “Refastenable adhesive fastening systems for individually packaged disposable absorbent articles” issued to Jennifer A. Roach et al. Examples of mechanical fastening system are also well known in the art. Examples include buttons, snaps, or hook and loop-type mechanical fasteners.

Thermal delivery surface 502 contains a heating element which is electrically connected to the treatment device. Preferably, thermal delivery surface 502 comprises a flexible material and flexible heating element also designed to conform to the irregular shape of nail plates. Examples of flexible heating elements include etch foil heaters, silicone rubber heaters, heat cables, fiberglass heating tapes, wire wound flexible heaters, electric heating tape, cable heaters, tape heaters.

The heating element of thermal delivery surface 502 of the embodiments is electrically connected to a treatment device similar to treatment device 100 of FIG. 1 by electrical connection 504. This electrical connection may be achieved, for example, by using a heater port in the housing like data port 132 of FIG. 1. Alternatively, nail cover 112 and thermal delivery surface 113 of FIG. 1 could be removed and replaced with flexible strap 500. The treatment device provides electrical current to thermal delivery surface 502 that produces heat through electrical resistance. The temperature of thermal delivery surface 502 is monitored by temperature sensor, not shown, which may be a thermistor or other electrical device that develops and regulates heat. The treatment device is able to adjust the power provided to thermal delivery surface 502 so as to maintain thermal delivery surface 502 at or near a set temperature and time chosen for the treatment.

Referring now to FIG. 6, an electrical block diagram showing an embodiment of the electrical system of treatment device 100 of FIG. 1 is shown. Treatment device 100 includes thermal delivery surface components mounted on circuit board 610. Thermal delivery surface components on circuit board 610 include the electrical components used to perform the treatment mounted on its surface. Circuit board 610 contains resistors, thermistors and other control components to develop and regulate heat. Resistors 612 mounted onto circuit board 610 are used to convert electrical energy from power source to heat energy needed to increase the temperature of thermal delivery surface 113, shown in FIG. 1. Control of the temperature of thermal delivery surface 113 is done in response to signals from temperature sensor 614, mounted on circuit board 610. Temperature sensor 614 provides an electrical signal indicative of the temperature of thermal delivery surface 113 to microprocessor 620 in housing 118 of FIG. 1.

A memory element 616 may also mounted on circuit board 610. Memory element 616 can be any combination of processing and memory elements utilized to store and implement thermal delivery surface specific functions. Memory element 616 of the embodiments is used to store thermal delivery surface specific information. For example, memory element 616 of the illustrated embodiment may include calibration information for its associated thermal delivery surface. As the individual components used in particular thermal delivery surface may have their own variances from their marked values, each thermal delivery surface is calibrated during manufacturing to provide calibration information stored in memory element 616 and used to adjust the heating algorithm of treatment device 100 to account for the particular values of the components in the thermal delivery surface.

The memory element 616 can also store treatment variables such as treatment cycle duration, treatment temperature and treatment frequency, as well as other information that aids the treatment device in its operation. Such information can, for example, be information that identifies the type of thermal delivery surface and the intended treatment protocols, as well as algorithm information used during a treatment cycle.

An electrical diagram showing an embodiment of the electrical system 610 of treatment device 100 of FIG. 1 is also illustrated in FIG. 6. Microprocessor 620 is programmed to respond to and control the various inputs and outputs of treatment device 100 of FIG. 1. Microprocessor 620 receives input from power button 642, and in response operates to power-up or power-down the treatment device accordingly. Microprocessor 620 also receives input from treatment button 644 and operates to start or stop treatment based on input from treatment button 644. LEDs 646 are turned on and off by microprocessor 620 to communicate visual information to the user, while speaker 630 is controlled by microprocessor 620 to communicate audible information to the user.

Microprocessor 620 is also in electrical communication with thermal delivery surface 113 of FIG. 1. In operation according to a preferred embodiment, microprocessor 620 communicates with memory element 616 and exchanges information on thermal delivery surface cycles, calibration, treatment variations and other thermal delivery surface specific information. Microprocessor 620 also preferably receives the signal from temperature sensor 614 through interface 632. Using the signal from temperature sensor 614, microprocessor 620 is operable to control the temperature of thermal delivery surface 113 of nail cover 112 of FIG. 1 or thermal delivery surface 502 of FIG. 5, Microprocessor 620 of the illustrated embodiment is connected to the gate of field effect transistor (“FET”) 634, and by varying the voltage at the gate of FET 634 is able to control the amount of current flowing through resistors 612. The heat produced by resistors 612 is proportional to the amount of current passing through them. Thermal interlock 618, which call be a fuse having a maximum current rating chosen to prevent runaway overheating of resistors 612, provides a safety mechanism to ensure that the failure of temperature sensor 614 does not cause a dangerous operating temperature in the thermal delivery surface.

Microprocessor 620 of the embodiments is programmed with a control algorithm referred to as a proportional, integral, derivative or PID. A PID is a control algorithm which uses three modes of operation: the proportional action is used to dampen the system response, the integral corrects for droop, and the derivative prevents overshoot and undershoot. The PID algorithm implemented in microprocessor 620 operates to bring the thermal delivery surface 113 to the desired operating temperature as quickly as possible with minimal overshoot, and also operates to respond to changes in the temperature of thermal delivery surface 113 during the treatment cycle that are caused by the heat sink effect of the treatment area.

In addition to being connected to FET 634, resistors 612 are connected to battery 622 through thermal interlock 618. Battery 622, which can be comprised of one or more individual cells, is charged by battery charger 624 when battery charger 624 is connected to external power supply 626. External power supply 626 can be any type of power supply, but is normally an AC to DC converter connected between battery charger 624 and an ordinary wall outlet. According to embodiments, the output voltage of battery 622 is directly related to the amount of charge left in battery 622, therefore, by a monitoring the voltage across battery 622 microprocessor 620 can determine the amount of charge remaining in battery 622 and convey this information to the user using LEDs 646 or speaker 630. Other methods of determining battery voltages or charge for different battery technologies can also be used and are well within the scope of the present invention.

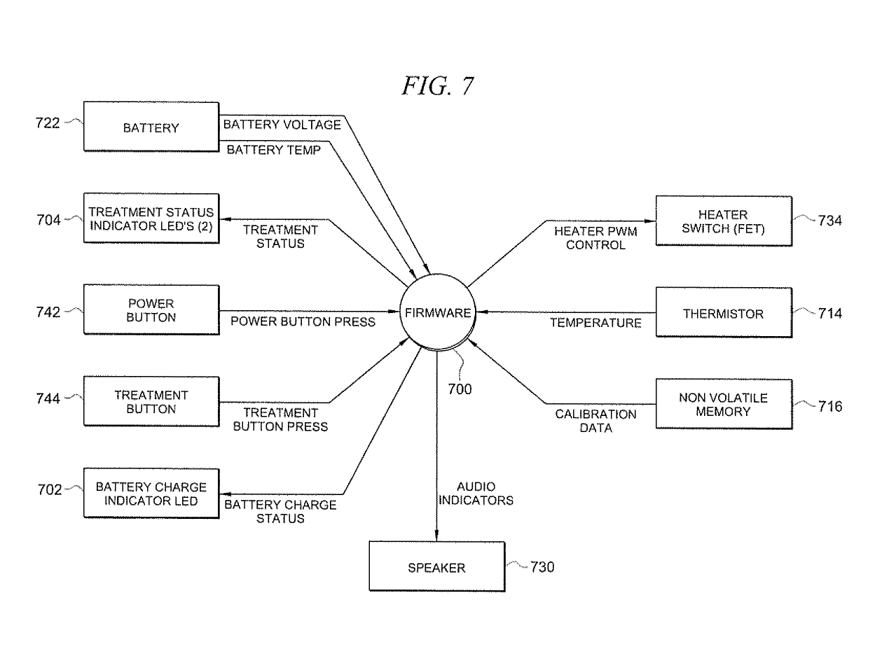

Referring now to FIG. 7, a diagram showing the various inputs to the firmware run by embodiments of microprocessor 620 of FIG. 6 is described. Firmware 700 represents the programming loaded on microprocessor 620. As described with reference to FIG. 6, microprocessor 620 is operable to respond to and control the various aspects of treatment device 100 of FIG. 1. Firmware 700 is able to accept inputs from power button 742, treatment button 744, temperature sensor 714 and battery 722. Firmware 700 is also able to exchange information with memory element 716, such as calibration data. The microprocessor 620 and memory element 716 may exchange any other information that may increase the efficacy of treatment device 100.

In response to the temperature sensor input and information from memory element 716, firmware 700 controls FET 734 to regulate the temperature of the thermal delivery surface according to the PID algorithm programmed into firmware 700. Firmware 700 also controls speaker 730 to provide audible feedback to the user and LEDs 702 and 704 which are subsets of LEDs 646 from FIG. 6, and provide indications of battery charge (LED 702) and treatment status (LEDs 704).

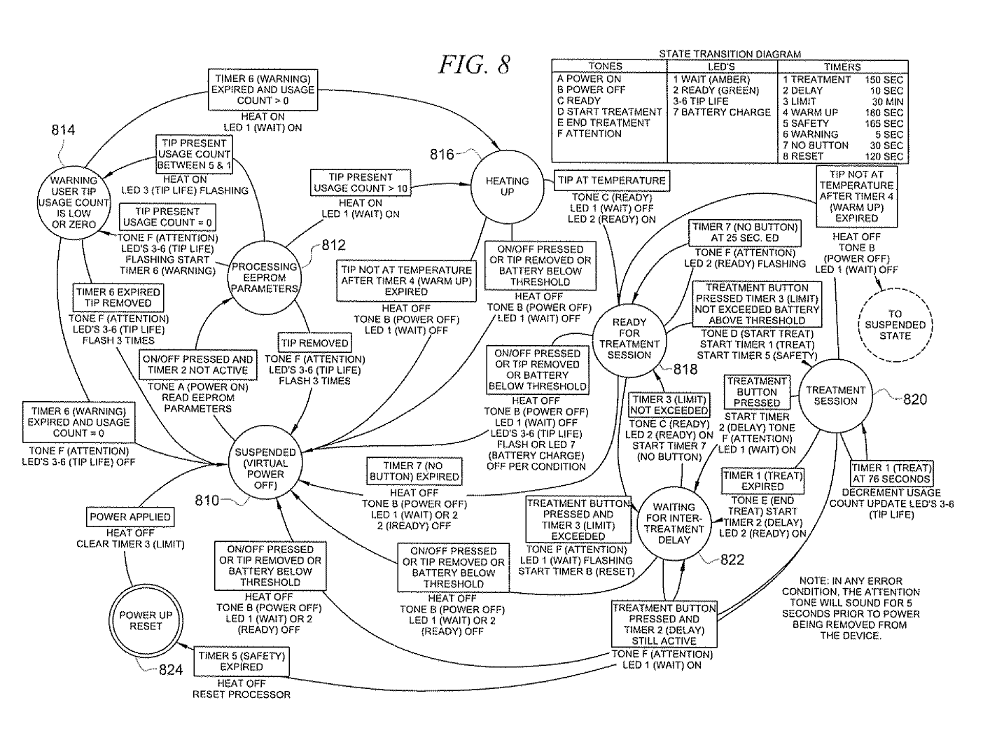

Referring now to FIG. 8, a state transition diagram showing various operating states of firmware 700 from FIG. 7 according to an embodiment is described. The state diagram begins a Suspended state 810 which is the power off state. During the power off mode the microprocessor is still receiving some power to allow it to monitor the power button. The Suspended state 810 is left when the power on button is pressed, and the state proceeds to the Processing Thermal delivery Surface Memory state 812. In the Processing Thermal delivery Surface Memory state 812 the microprocessor 630 and memory element 616 from FIG. 6 exchange thermal delivery surface specific treatment information. If the thermal delivery surface usage count is not low or zero, the state passes to Heating state 816. If the thermal delivery surface usage count is found to be low or zero the state progresses to the Warning state 814, which provides visual and or audible signals to the user to indicate that the thermal delivery surface usage count is low or zero. If the thermal delivery surface usage count is zero or the thermal delivery surface is removed, the state passes from the Warning state 814 to the Suspended state 810. If the thermal delivery surface uses count is low, but not zero the state passes from the Warning state 814 to the Heating state 816.

During the heating state 816 the thermal delivery surface is heated using resistors 612 from FIG. 6. A predictive model is used to set a timer based on the amount of time that should be required for the thermal delivery surface to come to temperature. This timer acts as in indicator that the thermal mass is responding to the heating correctly. If the thermal delivery surface does not reach the predetermined operating temperature by the expiration of the timer, it is an indication of a potentially faulty component and the treatment device shuts down by transitioning to Suspended state 810. Other predictions of thermal mass behavior can also be used to detect potentially faulty components.

In addition to the expiration of the timer, the treatment device powers down by transitioning to the Suspended state if the power button is pressed, or the battery voltage falls below a threshold, and indication of the fault is provided to the user through visual and/or audible signals. If the thermal delivery surface successfully reaches the operating temperature within the designated time the state transitions to Ready state 818. A timer is started upon entering the Ready state 818. If the timer expires or the power button is pressed while in the Ready state 818, the state transitions to the Suspended state 810.

If the treatment button is pressed while in Ready state 818 the state transitions to Treatment state 820. Two timers, a treatment timer and a safety timer, are started upon entering the Treatment state 820+The safety timer is slightly longer than the treatment timer so that if there is a failure in the treatment timer the safety timer will expire and transition the state to the Power Reset state 824 before transitioning to the Suspended state 810. The state also transitions from Treatment state 820 to Suspended state 810 if the power button is pressed during a treatment cycle.

As a treatment cycle can be a relatively long period of time, the treatment device can also be programmed to provide visual and/or audible indications of the progress of the treatment timer. For example, speaker 630 of FIG. 6 can be used to provide intermittent tones during the treatment to let the user know that the treatment is continuing. The time between the tones could be spaced to provide an indication of the remaining time in the treatment cycle, such as by shortening the time between the tones as the cycle gets closer to the end. Many other methods of providing visual or audible feedback could be contemplated and are well within the scope of the present invention.

When the treatment timer expires, or if the treatment button is pressed, the state transitions from Treatment state 820 to Wait state 822 which forces an inter-treatment delay. If the power button is pressed or the thermal delivery surface removed during the Wait state, the state transitions to Suspended state 810. After the expiration of the inter-treatment delay the state transitions back to Ready state 818. In addition to the inter-treatment delay, the Wait state 822 can be used to force a temporal treatment limit. While the inter-treatment delay forces a relatively brief delay between treatment cycles, the temporal treatment limit acts to limit the number of treatments that can be performed in specified period. For example, if the treatment cycle is two and a half minutes and the inter-treatment delay is 10 seconds, a temporal treatment limit of 30 minutes could be used to limit the device to approximately 10 to 11 consecutive treatments before a forced interval is imposed.

In another embodiment of the treatment device 100, an antibacterial, antiseptic, or antifungal compound may be introduced at air gap between the nail plate and nail bed, adding to the killing effect provided by the thermal energy. In turn, the heat created by thermal delivery surface 113 will aid in the dispersing and absorbing of such compounds creating a synergistic effect.

Although the preferred embodiments described above disclose the use of a temperature sensor which measures the temperature of the thermal deliver surface, other locations of the temperature sensor are well within the scope of this invention. Other locations include a temperature sensor located in the thermal transfer medium, such as thermal gel pack 502 of FIG. 2. By locating the temperature sensor in thermal gel pack 502, the temperature of the thermal gel pack could be used to control the thermal energy transferred to the nail bed infection. If the nail plate 402 has separated from the nail bed 404, such as shown in 4B, a temperature sensor could be located in the thermal gel 408 filling the air gap. Additionally, pseudo sensors are also possible. For example, the temperature of the thermal contact surface or thermal transfer medium could be predicted based on measurement of the power supplied to the heating element and the physical properties of the heating element.

Preferred Set Temperature and Treatment Time

To determine the preferred set temperature and treatment time, two factors should be considered. First, the set temperature and treatment time should be sufficient to cause a thermal reaction or response in the fungus, virus or bacteria detrimentally affecting the skin surface. Second, the set temperature and treatment time should be below the threshold that would damage the skin being treated. The first factor is discussed with reference to Examples 1-3 below using exemplary infectious agents. Based on Examples 1-3 a set temperature of 121 degrees Fahrenheit (49.44 degrees Celsius) for a period of 150 seconds proves to be effective for a variety of infectious agent and irritants. While a set temperature of 121 degrees Fahrenheit and a treatment time of 150 seconds are chosen for an embodiment of the present invention, other embodiments using combinations of set temperatures and treatment times which depart significantly from the described embodiment are well within the scope of the present invention.

To ensure that the described embodiment of a set time and temperature do not cause burn damage to the treatment area, modeling can be performed against previous research done into burn injuries. The modeling assumes that the skin surface in contact with the applicator immediately reaches the applicator temperature of 121 degrees Fahrenheit and remains at that temperature for the entire 150 seconds. First, the set temperature and treatment time are plotted against the Time-Surface Temperature Thresholds plot represented in FIG. 4, page 711 from Moritz and Henriques, “Studies of Thermal Energy,” American Journal of Pathology, 1947, Vol. 23, pp. 695-720, the disclosure of which is incorporated by reference. The plot of 49.44 degrees Celsius at 150 seconds is just under the dashed curve representing “the first morphological evidence of thermal damage,” such as slight reddening. At the set temperature, the curve indicates that the first reversible damage occurs at 195 seconds. Thus, according to Moritz and Henriques, the set temperature and treatment time are safe, and it worse might produce slight reddening of the treatment area.

Based on the data of Moritz and Henriques cited above, Xu and Qian in an article entitled “Analysis of Thermal Injury Process Based on Enzyme Deactivation Mechanisms,” in Journal of Biomechanical Engineering, Transactions of the ASME, Vol. 117, pp. 462-465 (1995), the disclosure of which is incorporated by reference, developed an equation for a damage function, 6, based on enzyme deactivation concepts.

$$Ω = {\int }_{0}^{1} \frac{1 * {10}^{- 1} exp ( 100 z )}{1 + 8 * {10}^{4} exp ( - 195 z )} d t$$

where z=1-305.65/T ° K. and t is in seconds

In this model T=322.59° K. and is constant, therefore,

Ω=4.947*10⁻³*Δt=0.742 for 150 seconds.

### EXAMPLE 1

Temperature dependent death curves for P. acnes.

While the bacteria P. acnes is not normally present in the nail bed, nor the cause of onychomycosis, the reaction of P. acnes to heating can he considered illustrative of the expected reactions of those infection agents which are responsible for onychomycosis and other nail bed infections treatable by the device described herein.

Materials and Methods: The bacterial strain P. acnes was purchased from The American Type Culture Collection ATCC No. 11827, Lot 419571, Manassas, Va.). The cultures were stored in KWIK-STIK lyophilized preparations. The lyophillized cells (P. acnes) were rehydrated according to the manufacturers recommendations and initially grown on a streak plate to isolate individual colonies under anaerobic conditions. These plates were then incubated overnight at 37 degrees Celsius in an anaerobic chamber. Individual colonies were then isolated and inoculated into TSB-growth media with medium agitation overnight. From these aliquots of 0.1 ml of TSB broth culture was added to the 0.9 ml of PBS sterile buffer. This mixture was then transferred to thin-walled Eppendorf 1.5 ml tubes and placed in a heating block at various times and temperatures. The cultures after specific incubation times were removed and 0.1 ml of the material was plated onto TSA plates. This mixture was then spread with a sterile hockey-stick and then allowed to incubate at 37 degrees Celsius for 5 days in anaerobic conditions. The plates were then removed and colonies were counted and recorded. The results are demonstrated in FIG. 10. FIG. 10 demonstrates the rapid decline of P. acnes in response to various temperatures and duration of treatment. The baseline P. acnes colony count that had not been exposed to the heat source was 1050.

Results: A general trend of reduction of required time to kill the bacterial strain is seen at higher temperature incubations. Also of note is the temporal thermal threshold where the number of colonies drops off in a very steep fashion. By using the curves generated by such experiments the optimal thermal output and the timing for each temperature can be extrapolated for a localized heating device. The in vitro data shown demonstrates significant sensitivity of P. acnes bacterial cells to the effects of sustained low-level heat. Temperatures of 55 degrees Celsius result in the death of substantially all of the bacteria after 3½ minutes. Temperatures of 58 and 59 decrees Celsius result in the death of substantially all of the bacteria after 2 minutes. These curves demonstrate that P. acnes can be rendered largely non-viable by treatment under the conditions shown by the death curves.

### EXAMPLE 2

Again, though acne is a skin condition, the treatment of skin lesions using heat is considered to be illustrative of utility of heat treatment for onychomycosis and other nail bed infections using the concepts described herein.

Treatment of acne lesions in human subjects. The inventors have performed preliminary studies on over 100 volunteers experiencing outbreaks of acne lesions. All subjects reported being satisfied with the results obtained. The results showed a clear response to treatment in approximately 90% of subjects treated. No subject reported any serious adverse effects due to treatment. Furthermore, we have discovered that a treated lesion heals more than 80% faster than untreated lesions.

The electrical device used in the present study had an interface of approximately 0.4 cm². The interface of the device was heated to a constant temperature of approximately 48-50 degrees Celsius prior to application of the device to the skin surface, and the temperature was maintained during the treatment period. Each of the subjects was given instructions on how to use the device and was monitored during the treatment. The treatment consisted of a 2½ minute application of the device to the lesion site. The study called for the application of two treatment cycles to each patient, with the second treatment cycle being administered 12 hours after the first. In practice, however, the treatments were frequently only conducted once on each subject because twelve hours after the first treatment many of the lesions had healed to an extent that they did not require any further treatment.

Results of experiments performed on volunteer subjects are listed in Table 1. Members of the control group were not treated. Members of the treatment group were treated as described above. Both groups either examined or self-reported the results of treatment over the following 14 days. Only results from study participants who reported data for 14 days were included in the table. The data is reported in terms of the size of the lesion prior to treatment. A lesion size of 100% indicates that the lesion size was unchanged. Lesion size was approximated in increments of 10%. A lesion size of 0% indicates that the lesion had fully healed.

TABLE 1

| | | | | Day | Day | Day | Day | Day | Day | Day | Day | Day | Day | Day | Day | Day | Day |
|---|---|---|---|---|---|---|---|---|---|---|---|---|---|---|---|---|---|
| # | Name | Gender | Age | 1 | 2 | 3 | 4 | 5 | 6 | 7 | 8 | 9 | 10 | 11 | 12 | 13 | 14 |

Control Group

| | | | | | | | | | | | | | | | | | |
|---|---|---|---|---|---|---|---|---|---|---|---|---|---|---|---|---|---|
| 1 | LEF | F | 27 | 100% | 100% | 100% | 100% | 90% | 90% | 80% | 80% | 50% | 20% | 10% | 0% | 0% | 0% |
| 2 | AMC | F | 22 | 100% | 100% | 100% | 90% | 90% | 80% | 80% | 60% | 40% | 40% | 20% | 20% | 20% | 10% |
| 3 | AWC | F | 16 | 100% | 100% | 100% | 100% | 100% | 100% | 100% | 80% | 80% | 60% | 40% | 10% | 10% | 10% |
| 4 | KAC | F | 13 | 100% | 100% | 100% | 80% | 80% | 70% | 40% | 40% | 40% | 40% | 20% | 10% | 0% | 0% |
| 5 | ECP | F | 35 | 100% | 100% | 100% | 100% | 80% | 80% | 80% | 20% | 20% | 20% | 20% | 10% | 0% | 0% |
| 6 | KSL | F | 21 | 100% | 100% | 90% | 90% | 80% | 80% | 60% | 60% | 60% | 30% | 30% | 10% | 10% | 0% |
| 7 | NET | F | 18 | 100% | 100% | 100% | 80% | 80% | 80% | 60% | 60% | 60% | 30% | 30% | 30% | 10% | 10% |
| 8 | LHJ | F | 27 | 100% | 100% | 100% | 80% | 80% | 80% | 50% | 50% | 50% | 50% | 20% | 10% | 10% | 0% |
| 9 | TAA | F | 28 | 100% | 90% | 90% | 90% | 90% | 70% | 70% | 70% | 40% | 30% | 30% | 10% | 10% | 10% |
| | | | Total | 100% | 99% | 98% | 90% | 86% | 81% | 69% | 58% | 49% | 36% | 24% | 12% | 8% | 4% |
| 1 | ZAC | M | 15 | 100% | 100% | 100% | 100% | 80% | 80% | 60% | 60% | 60% | 40% | 30% | 30% | 10% | 0% |
| 2 | ZMP | M | 14 | 100% | 100% | 100% | 100% | 90% | 90% | 90% | 80% | 80% | 60% | 60% | 20% | 20% | 10% |
| 3 | MAP | M | 18 | 100% | 100% | 100% | 100% | 90% | 90% | 90% | 70% | 70% | 70% | 30% | 30% | 10% | 0% |
| 4 | CDC | M | 40 | 100% | 100% | 90% | 80% | 70% | 70% | 30% | 30% | 30% | 10% | 10% | 0% | 0% | 0% |
| 5 | CAC | M | 24 | 100% | 100% | 100% | 90% | 80% | 80% | 80% | 50% | 50% | 50% | 20% | 20% | 10% | 0% |
| 6 | RAA | M | 33 | 100% | 100% | 100% | 90% | 80% | 70% | 70% | 60% | 60% | 40% | 20% | 20% | 10% | 10% |
| | | | Total | 100% | 100% | 98% | 93% | 82% | 80% | 70% | 58% | 58% | 45% | 28% | 20% | 10% | 3% |
| | | | Total | 100% | 99% | 98% | 91% | 84% | 81% | 69% | 58% | 53% | 39% | 26% | 15% | 9% | 4% |

Treatment Group

| | | | | | | | | | | | | | | | | | |
|---|---|---|---|---|---|---|---|---|---|---|---|---|---|---|---|---|---|
| 1 | AAS | F | 34 | 100% | 30% | 20% | 10% | 0% | 0% | 0% | 0% | 0% | 0% | 0% | 0% | 0% | 0% |
| 2 | ACC | F | 36 | 100% | 20% | 0% | 0% | 0% | 0% | 0% | 0% | 0% | 0% | 0% | 0% | 0% | 0% |
| 3 | AWC | F | 40 | 100% | 70% | 30% | 10% | 0% | 0% | 0% | 0% | 0% | 0% | 0% | 0% | 0% | 0% |
| 4 | BAB | F | 27 | 100% | 10% | 0% | 0% | 0% | 0% | 0% | 0% | 0% | 0% | 0% | 0% | 0% | 0% |
| 5 | CAB | F | 29 | 100% | 0% | 0% | 0% | 0% | 0% | 0% | 0% | 0% | 0% | 0% | 0% | 0% | 0% |
| 6 | CHH | F | 30 | 100% | 60% | 60% | 40% | 10% | 0% | 0% | 0% | 0% | 0% | 0% | 0% | 0% | 0% |
| 7 | DSF | F | 33 | 100% | 0% | 0% | 0% | 0% | 0% | 0% | 0% | 0% | 0% | 0% | 0% | 0% | 0% |
| 8 | GDL | F | 34 | 100% | 40% | 10% | 0% | 0% | 0% | 0% | 0% | 0% | 0% | 0% | 0% | 0% | 0% |
| 9 | HCD | F | 14 | 100% | 50% | 20% | 0% | 0% | 0% | 0% | 0% | 0% | 0% | 0% | 0% | 0% | 0% |
| 10 | HLL | F | 36 | 100% | 0% | 0% | 0% | 0% | 0% | 0% | 0% | 0% | 0% | 0% | 0% | 0% | 0% |
| 11 | JLP | F | 19 | 100% | 20% | 0% | 0% | 0% | 0% | 0% | 0% | 0% | 0% | 0% | 0% | 0% | 0% |
| 12 | JSH | F | 28 | 100% | 20% | 20% | 0% | 0% | 0% | 0% | 0% | 0% | 0% | 0% | 0% | 0% | 0% |
| 13 | JUL | F | 31 | 100% | 70% | 50% | 30% | 10% | 0% | 0% | 0% | 0% | 0% | 0% | 0% | 0% | 0% |
| 14 | KAC | F | 13 | 100% | 50% | 30% | 10% | 0% | 0% | 0% | 0% | 0% | 0% | 0% | 0% | 0% | 0% |
| 15 | KDJ | F | 20 | 100% | 20% | 0% | 0% | 0% | 0% | 0% | 0% | 0% | 0% | 0% | 0% | 0% | 0% |
| 16 | KEF | F | 26 | 100% | 10% | 0% | 0% | 0% | 0% | 0% | 0% | 0% | 0% | 0% | 0% | 0% | 0% |
| 17 | KFC | F | 17 | 100% | 0% | 0% | 0% | 0% | 0% | 0% | 0% | 0% | 0% | 0% | 0% | 0% | 0% |
| 18 | KST | F | 33 | 100% | 80% | 80% | 60% | 30% | 10% | 0% | 0% | 0% | 0% | 0% | 0% | 0% | 0% |
| 19 | LEF | F | 21 | 100% | 30% | 10% | 10% | 0% | 0% | 0% | 0% | 0% | 0% | 0% | 0% | 0% | 0% |
| 20 | LKD | F | 34 | 100% | 50% | 50% | 50% | 30% | 30% | 20% | 10% | 10% | 0% | 0% | 0% | 0% | 0% |
| 21 | LKJ | F | 15 | 100% | 70% | 40% | 20% | 10% | 0% | 0% | 0% | 0% | 0% | 0% | 0% | 0% | 0% |
| 22 | MDD | F | 35 | 100% | 20% | 0% | 0% | 0% | 0% | 0% | 0% | 0% | 0% | 0% | 0% | 0% | 0% |
| 23 | MDF | F | 19 | 100% | 50% | 10% | 0% | 0% | 0% | 0% | 0% | 0% | 0% | 0% | 0% | 0% | 0% |
| 24 | MEA | F | 38 | 100% | 70% | 30% | 20% | 20% | 10% | 0% | 0% | 0% | 0% | 0% | 0% | 0% | 0% |
| 25 | MLJ | F | 29 | 100% | 60% | 30% | 10% | 0% | 0% | 0% | 0% | 0% | 0% | 0% | 0% | 0% | 0% |
| 26 | NJM | F | 37 | 100% | 50% | 40% | 10% | 0% | 0% | 0% | 0% | 0% | 0% | 0% | 0% | 0% | 0% |
| 27 | RTY | F | 23 | 100% | 10% | 0% | 0% | 0% | 0% | 0% | 0% | 0% | 0% | 0% | 0% | 0% | 0% |
| 28 | SAH | F | 18 | 100% | 40% | 10% | 0% | 0% | 0% | 0% | 0% | 0% | 0% | 0% | 0% | 0% | 0% |
| 29 | SAL | F | 14 | 100% | 50% | 10% | 0% | 0% | 0% | 0% | 0% | 0% | 0% | 0% | 0% | 0% | 0% |
| 30 | SBH | F | 18 | 100% | 20% | 20% | 10% | 0% | 0% | 0% | 0% | 0% | 0% | 0% | 0% | 0% | 0% |
| 31 | SFH | F | 35 | 100% | 0% | 0% | 0% | 0% | 0% | 0% | 0% | 0% | 0% | 0% | 0% | 0% | 0% |
| 32 | SLB | F | 31 | 100% | 60% | 30% | 30% | 10% | 100% | 0% | 0% | 0% | 0% | 0% | 0% | 0% | 0% |
| 33 | TCA | F | 16 | 100% | 0% | 0% | 0% | 0% | 0% | 0% | 0% | 0% | 0% | 0% | 0% | 0% | 0% |
| 34 | TDB | F | 25 | 100% | 20% | 0% | 0% | 0% | 0% | 0% | 0% | 0% | 0% | 0% | 0% | 0% | 0% |
| 35 | TEM | F | 38 | 100% | 60% | 30% | 30% | 10% | 10% | 10% | 0% | 0% | 0% | 0% | 0% | 0% | 0% |
| 36 | TLS | F | 13 | 100% | 80% | 40% | 20% | 10% | 10% | 10% | 0% | 0% | 0% | 0% | 0% | 0% | 0% |
| 37 | TSJ | F | 36 | 100% | 50% | 30% | 10% | 10% | 0% | 0% | 0% | 0% | 0% | 0% | 0% | 0% | 0% |
| 38 | VYM | F | 21 | 100% | 80% | 80% | 80% | 50% | 30% | 10% | 10% | 10% | 0% | 0% | 0% | 0% | 0% |
| | | | Total | 100% | 37% | 21% | 12% | 5% | 5% | 1% | 1% | 1% | 0% | 0% | 0% | 0% | 0% |
| 1 | CAC | M | 40 | 100% | 20% | 10% | 0% | 0% | 0% | 0% | 0% | 0% | 0% | 0% | 0% | 0% | 0% |
| 2 | CDM | M | 39 | 100% | 60% | 40% | 10% | 10% | 0% | 0% | 0% | 0% | 0% | 0% | 0% | 0% | 0% |
| 3 | DAD | M | 16 | 100% | 20% | 10% | 0% | 0% | 0% | 0% | 0% | 0% | 0% | 0% | 0% | 0% | 0% |
| 4 | DDL | M | 21 | 100% | 0% | 0% | 0% | 0% | 0% | 0% | 0% | 0% | 0% | 0% | 0% | 0% | 0% |
| 5 | DFB | M | 35 | 100% | 80% | 80% | 40% | 20% | 10% | 10% | 10% | 10% | 0% | 0% | 0% | 0% | 0% |
| 6 | EHE | M | 14 | 100% | 20% | 0% | 0% | 0% | 0% | 0% | 0% | 0% | 0% | 0% | 0% | 0% | 0% |
| 7 | HAF | M | 33 | 100% | 60% | 60% | 20% | 20% | 10% | 10% | 0% | 0% | 0% | 0% | 0% | 0% | 0% |
| 8 | JEY | M | 15 | 100% | 20% | 20% | 10% | 0% | 0% | 0% | 0% | 0% | 0% | 0% | 0% | 0% | 0% |
| 9 | JKG | M | 18 | 100% | 40% | 10% | 10% | 0% | 0% | 0% | 0% | 0% | 0% | 0% | 0% | 0% | 0% |
| 10 | KEG | M | 36 | 100% | 0% | 0% | 0% | 0% | 0% | 0% | 0% | 0% | 0% | 0% | 0% | 0% | 0% |
| 11 | KSP | M | 31 | 100% | 30% | 30% | 10% | 10% | 0% | 0% | 0% | 0% | 0% | 0% | 0% | 0% | 0% |
| 12 | MJP | M | 34 | 100% | 20% | 20% | 10% | 0% | 0% | 0% | 0% | 0% | 0% | 0% | 0% | 0% | 0% |
| 13 | OAP | M | 20 | 100% | 90% | 40% | 20% | 10% | 0% | 0% | 0% | 0% | 0% | 0% | 0% | 0% | 0% |
| 14 | PLT | M | 38 | 100% | 70% | 50% | 30% | 10% | 10% | 0% | 0% | 0% | 0% | 0% | 0% | 0% | 0% |
| 15 | RAA | M | 21 | 100% | 20% | 20% | 0% | 0% | 0% | 0% | 0% | 0% | 0% | 0% | 0% | 0% | 0% |
| 16 | RDC | M | 30 | 100% | 30% | 10% | 10% | 0% | 0% | 0% | 0% | 0% | 0% | 0% | 0% | 0% | 0% |
| 17 | RCJ | M | 25 | 100% | 60% | 20% | 20% | 20% | 10% | 0% | 0% | 0% | 0% | 0% | 0% | 0% | 0% |
| 18 | TFL | M | 16 | 100% | 0% | 0% | 0% | 0% | 0% | 0% | 0% | 0% | 0% | 0% | 0% | 0% | 0% |
| 19 | SHT | M | 28 | 100% | 20% | 10% | 0% | 0% | 0% | 0% | 0% | 0% | 0% | 0% | 0% | 0% | 0% |
| 20 | DKP | M | 36 | 100% | 50% | 10% | 10% | 0% | 0% | 0% | 0% | 0% | 0% | 0% | 0% | 0% | 0% |
| 21 | WRT | M | 28 | 100% | 30% | 10% | 0% | 0% | 0% | 0% | 0% | 0% | 0% | 0% | 0% | 0% | 0% |
| 22 | WJK | M | 32 | 100% | 80% | 80% | 60% | 40% | 40% | 20% | 20% | 10% | 10% | 0% | 0% | 0% | 0% |
| 23 | PLL | M | 24 | 100% | 20% | 0% | 0% | 0% | 0% | 0% | 0% | 0% | 0% | 0% | 0% | 0% | 0% |
| 24 | MWT | M | 31 | 100% | 0% | 0% | 0% | 0% | 0% | 0% | 0% | 0% | 0% | 0% | 0% | 0% | 0% |
| 25 | TTM | M | 26 | 100% | 10% | 10% | 0% | 0% | 0% | 0% | 0% | 0% | 0% | 0% | 0% | 0% | 0% |
| 26 | BTL | M | 37 | 100% | 60% | 30% | 10% | 10% | 0% | 0% | 0% | 0% | 0% | 0% | 0% | 0% | 0% |
| 27 | DWD | M | 22 | 100% | 70% | 20% | 20% | 10% | 0% | 0% | 0% | 0% | 0% | 0% | 0% | 0% | 0% |
| | | | Total | 100% | 36% | 22% | 11% | 6% | 3% | 1% | 1% | 1% | 0% | 0% | 0% | 0% | 0% |
| | | | Total | 100% | 37% | 21% | 11% | 6% | 4% | 1% | 1% | 1% | 0% | 0% | 0% | 0% | 0% |

### EXAMPLE 3

The inventors have tested prototype devices on multiple oral herpes lesions of human volunteers, and the results have shown a complete termination of the herpetic lesion after two applications of the device at 2½ minutes per treatment, 12 hours apart as described in Example 2. The volunteers reported a marked decrease in healing time after treatment versus the usual healing cycle for lesions of this type.

All of the compositions and methods disclosed and claimed herein can be made and executed without undue experimentation in light of the present disclosure. While the compositions and methods of this invention have been described in terms of preferred embodiments, it will be apparent to those of skill in the art that variations can be applied to the devices or methods and in the steps or in the sequence of steps of the methods described herein without departing from the concept, spirit and scope of the invention. More specifically, it will be apparent that certain mechanical elements related to those described above can be substituted for the mechanical elements described herein to achieve the same or similar results. All such similar substitutes and modifications apparent to those skilled in the art are deemed to be within the spirit, scope and concept of the invention as defined by the appended claim.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A device for the heat treatment of nails comprising:
at least one thermal delivery surface adapted to be placed in contact with the nail plate; and
a heating element operable to heat the thermal delivery surface to a treatment temperature.

2. The device in claim 1, where in the treatment temperature is within a temperature range selected for combating the fungus, bacteria, or viruses known to contribute to nail bed infections.

3. The device in claim 1, wherein the treatment temperature is in a range of about 45 degrees Celsius to about 65 degrees Celsius.

4. The device in claim 1, wherein the nail bed infection is a fungal infection.

5. The device in claim 17 wherein the nail bed infection is a dermatophyte infection.

6. The device in claim 1, wherein the nail bed infection is a bacterial infection.

7. The device in claim 17 wherein the nail bed infection is a viral infection.

8. The device in claim 1, wherein the nail bed infection is a mold infection.

9. The device in claim 1, wherein the nail bed infection is a non-dermatophyte yeast infection.

10. The device in claim 1, further comprising a spring wherein the spring is operable to apply a downward pressure on the thermal delivery surface.

11. The device in claim 1, wherein the thermal delivery surfaces is integrated in one or more flexible straps.

12. The device in claim 11, wherein the flexible strap is held in place by an adhesive fastening system.

13. The device in claim 11, wherein the flexible strap is held in place by a mechanical fastening system.

14. The device of claim 1 further comprising:
a temperature sensor; and
a controller electrically connected to the heating element and the temperature sensor, wherein the controller is operable to control the heating element in response to a signal from the temperature sensor and regulate the temperature of the thermal delivery surface to the treatment temperature.

15. The device in claim 10, wherein the controller comprises a microprocessor.

16. The device in claim 10, wherein the controller comprises a timer.

17. The device in claim 12, wherein the timer operates between 10 seconds and 30 minutes.

18. The device in claim 10, wherein the controller includes a PID algorithm for temperature control.

19. The device in claim 10, wherein the controller comprises a separate housing and is connected to the thermal delivery surface by wire leads.

20. The device in claim 18 wherein the separate housing comprises a user interface.

21. The device in claim 1, further comprising a thermal transfer medium to aid the transfer of heat from the thermal delivery surface to the nail plate.

22. The device in claim 21, wherein the thermal transfer medium is adapted to fit over the nail plate.

23. The device in claim 22, wherein the thermal transfer medium further comprises a deformable thermally conductive material, the deformable material able to deform when placed in contact with the patent's nail plate to provided improved transfer of thermal energy from the thermal delivery surface to the nail plate.

24. The device in claim 21, further comprising a thermal gel, the thermal gel able to fill the air gap formed between the nail plate and the nail bed to provide improved transfer of thermal energy from the thermal delivery surface to the nail plate.

25. The device in claim 24 wherein the thermal gel further comprises an anti-fungal agent.

26. A method of treating or preventing nail bed infection by application of heat, the method comprising:
applying, a thermal delivery surface to the nail plate;
heating the thermal delivery surface to transfer heat to a nail bed infection at a temperature selected for treatment of a the nail bed infection; and
raising the temperature of the nail plate to the temperature for a predetermined period of time to provide the treatment to the nail bed infection.

27. The method recited in claim 26, wherein the nail bed infection is a fungal infection.

28. The method recited in claim 26, wherein the nail bed infection is a dermatophyte infection.

29. The method recited in claim 26, wherein the nail bed infection is a bacterial infection.

30. The method recited in claim 26, wherein the nail bed infection is a viral infection.

31. The method recited in claim 26, wherein the nail bed infection is a mold infection.

32. The method recited in claim 26, wherein the nail bed infection is a non-dermatophyte yeast infection.

33. The method of claim 26, where in the treatment temperature is within a temperature range capable of combating the fungus, bacteria, or viruses known to contribute to nail bed infections.

34. The method of claim 26, wherein the temperature is a temperature between about 46 decrees Celsius and about 65 degrees Celsius.

35. The method of claim 26, further comprising applying a thermal transfer medium to the nail plate to aid in the transfer of thermal energy from the thermal delivery surface.

36. The method of claim 35, further comprising applying a thermal gel between the nail bed and nail plate.

37. The method of claim 35, wherein the thermal gel further comprises an anti-fungal agent.

38. The method of claim 26, further comprising indicating operation information visually and aurally

39. A method of treating or preventing nail bed infection by application of heat, the method comprising:
selecting the size and shape of a thermal delivery surface to correspond to the size and shape of the nail to be treated;
applying the thermal delivery surface to the nail plate.
removably connecting a heating element to a controller;
heating the thermal delivery surface to transfer heat to a nail bed infection at a temperature selected for treatment of a the nail bed infection; and
raising the temperature of the nail plate to the temperature for a predetermined period of time to provide the treatment to the nail bed infection.

40. The method of claim 39, wherein the treatment temperature is within a temperature range selected for combating the fungus, bacteria, or viruses known to contribute to nail bed infections.

41. The method of claim 39, wherein the treatment temperature is in a range of about 45 degrees Celsius to about 65 degrees Celsius.

42. The method of claim 39, wherein the nail bed infection is a fungal infection.

43. The method of claim 39, wherein the nail bed infection is a dermatophyte infection.

44. The method of claim 39, wherein the nail bed infection is a bacterial infection.

45. The method of claim 39, wherein the nail bed infection is a viral infection.

46. The device in claim 1, wherein the nail bed infection is a mold infection.

47. The device in claim 1, wherein the nail bed infection is a non-dermatophyte yeast infection.
